(19) United States
(12) Patent Application Publication
(10) Pub. No.: US 2016/0213030 A1
(43) Pub. Date: Jul. 28, 2016

(54) EDIBLE COATING COMPOSITIONS, EDIBLE COATINGS, AND METHODS FOR MAKING AND USING THE SAME

(71) Applicant: Sensient Colors LLC, St. Louis, MO (US)

(72) Inventor: Beverly A. Schad, Union, MO (US)

(21) Appl. No.: 14/915,233

(22) PCT Filed: Aug. 28, 2014
(86) PCT No.: PCT/US14/53240

§ 371 Date: Feb. 27, 2016

Related U.S. Application Data
(60) Provisional application No. 61/871,282, filed on Aug. 28, 2013.
(60) Provisional application No. 61/873,346, filed on Sep. 3, 2013.

(51) Int. Cl. A61K 9/28; A61K 47/38; A61K 47/14; A61K 47/12; A61K 9/00; A61K 47/24; A23L 1/0534

(52) U.S. Cl. CPC A23L 1/0052; A61K 9/0053; A61K 47/38; A61K 47/14; A61K 47/24; A61K 47/12; A23V 2002/00

(57) ABSTRACT

Edible coating compositions with at least one of hydroxypropyl methylcellulose, a triester of citric acid, lecithin, and a C12-24 fatty acid. The edible coating compositions may be used in the food, pharmaceutical and nutraceutical industries. This disclosure also provides a method of making a coating composition, which comprises combining hydroxypropyl methylcellulose, a triester of citric acid, lecithin, and a C12-24 fatty acid to form a mixture; and optionally combining the mixture with water.

### CROSS-REFERENCE TO RELATED APPLICATIONS

This application claims priority to U.S. Provisional Application No. 61/871,282 filed on Aug. 28, 2013 and U.S. Provisional Application No. 61/873,345 filed on Sep. 3, 2013, both of which are incorporated fully herein by reference.

### FIELD OF USE

The present invention relates to edible coating compositions, edible coatings, and methods for making and using the same, for use in the food, pharmaceutical and nutraceutical industries.

### INTRODUCTION

Traditionally, coatings comprising hydroxypropyl methylcellulose (HPMC) have exhibited moisture barrier properties. However, it is believed that some portion of the moisture barrier properties are provided by virtue of the coating absorbing water (moisture uptake by the coating), and thus preventing moisture from penetrating the coating to reach the core. However, this absorption of water causes swelling of the coating and limits the useful lifetime of the coating, due to the coating possessing a finite capacity to absorb water. Furthermore, the effectiveness of a moisture barrier based on absorbing water within the coating is directly linked to the thickness of the coating, whereas a coating that provides a moisture barrier without absorbing water can function at any thickness, so long as the coating is thick enough to prevent passage of water. As a simple analogy, the way a sorbent material such as a sponge would function at preventing passage of moisture is similar to the moisture barrier coating based on absorbing water and the way a waterproof material such as a sheet of plastic would function at preventing passage of moisture is similar to a moisture barrier with reduced moisture uptake. Accordingly, a need exists for a coating comprising HPMC having a moisture uptake that is reduced compared to conventional HPMC coatings.

### SUMMARY

This disclosure provides edible coating compositions comprising hydroxypropyl methylcellulose, a triester of citric acid, lecithin, and a C₁₂₋₂₄ fatty acid. The C₁₂₋₂₄ fatty acid may be present in an amount of less than about 32.5% by weight of the non-water components of the composition or the C₁₂₋₂₄ fatty acid may be present in an amount of from about 32.5% to about 50% by weight of the non-water components of the composition and the triester of citric acid is present in an amount of greater than about 4% by weight of the non-water components of the composition.

The disclosure may also provide an edible coating composition comprising about 50.0% to about 75.0% by weight hydroxypropyl methylcellulose; about 1.0% to about 6.0% by weight triester of citric acid; about 0.5% to about 10.0% by weight of lecithin; and about 15.0% to about 45.0% by weight of a C₁₂₋₂₄ fatty acid, wherein all percentages by weight are of non-water components of the composition. Applying the composition to an edible substrate to a weight gain of about 5% compared to the edible substrate may produce an edible substrate coated with an edible coating. The edible coating may have a moisture uptake of less than about 1% when exposed to 40° C. and 75% relative humidity for 30 minutes as measured by a VTI-SA Sorption Analyzer.

This disclosure also provides a method of making a coating composition, which comprises combining hydroxypropyl methylcellulose, a triester of citric acid, lecithin, and a C12-24 fatty acid to form a mixture; and optionally combining the mixture with water.

This disclosure also provides a coated edible substrate comprising a food, pharmaceutical or nutraceutical having an edible coating applied thereto. The edible coating may comprise hydroxypropyl methylcellulose, a triester of citric acid, lecithin and a C12-24 fatty acid. The C₁₂₋₂₄ fatty acid may be present in an amount of less than about 32.5% by weight of the non-water components of the composition or the C₁₂₋₂₄ fatty acid may be present in an amount of from about 32.5% to about 50% by weight of the non-water components of the composition and the triester of citric acid is present in an amount of greater than about 4% by weight of the non-water components of the composition.

This disclosure also provides a coated edible substrate comprising a food, pharmaceutical or nutraceutical having an edible coating applied thereto. The edible coating may comprise about 50.0% to about 75.0% by weight hydroxypropyl methylcellulose; about 1.0% to about 6.0% by weight triester of citric acid; about 0.5% to about 10.0% by weight of lecithin; and about 15.0% to about 45.0% by weight of a C12-24 fatty acid. The edible coating may be applied to a weight gain of about 3% compared to the food, pharmaceutical or nutraceutical, and the edible coating may have a moisture uptake of less than about 0.5% when exposed to 40° C. and 75% relative humidity for 30 minutes as measured by a VTI-SA Sorption Analyzer.

### BRIEF DESCRIPTION OF THE FIGURES

FIG. 1 shows swallowability-slip analysis for different coatings.

### DETAILED DESCRIPTION

Before any embodiments of the invention are explained in detail, it is to be understood that the invention is not limited in its application to the details of construction and the arrangement of components set forth in the following description. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. The present disclosure also contemplates other embodiments “comprising,” “consisting of” and “consisting essentially of,” the embodiments or elements presented herein, whether explicitly set forth or not.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application. It also is understood that for the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” is used synonymously with the term “approximately.” Illustratively, the use of the term “about” indicates that a recited value may include additional values slightly outside the recited values. This variation may be due to conditions such as experimental error, manufacturing tolerances, variations in equilibrium conditions, and the like. In some embodiments, the term “about” may include the cited value plus or minus 2.5%, 5%, 7.5%, or 10%, among others.

Disclosed herein are edible coating compositions comprising at least one of HPMC, a triester of citric acid (e.g., triethyl citrate), a lecithin (e.g., sunflower lecithin), a C₁₂₋₂₄ fatty acid (e.g., stearic acid), water, and combinations thereof. In one embodiment, the edible coating compositions comprise HPMC, triethyl citrate, lecithin, and stearic acid. The edible coating compositions may consist essentially of hydroxypropyl methylcellulose, a triester of citric acid, lecithin, a C₁₂₋₂₄ fatty acid, and optionally water. Also disclosed are methods of making edible coating compositions. Further disclosed are methods of using edible coating compositions including methods of making edible coatings. The edible coatings may comprise at least one of HPMC, a triester of citric acid (e.g., triethyl citrate), lecithin (e.g., sunflower lecithin), a C₁₂₋₂₄ fatty acid (e.g., stearic acid), and combinations thereof. In one embodiment, the edible coatings comprise HPMC, triethyl citrate, sunflower lecithin, and stearic acid. The edible coatings may consist essentially of hydroxypropyl methylcellulose, a triester of citric acid, lecithin, and a C₁₂₋₂₄ fatty acid.

The edible coating compositions can form edible coatings which may have improved properties when compared to conventional coatings that contain HPMC. In particular, the coatings may have at least one of reduced moisture uptake, improved stability, improved clarity or opacity, and combinations thereof compared to conventional HPMC coatings.

### 1. Edible Coating Compositions

The edible coating compositions of the present invention may comprise at least one of hydroxypropyl methycellulose, triester of citric acid (e.g., triethyl citrate), lecithin, a C₁₂₋₂₄ fatty acid (e.g., stearic acid), and optionally water. The compositions are useful for coating edible substrates. The compositions can be shipped in dry form or with any optional water added. The edible coating has enhanced uniformity, and no subcoating is required. The edible coatings may have enhanced uniformity.

a. Hydroxypropyl Methylcellulose

HPMC may be used to form the edible coating compositions. Examples of commercially-available HPMC may include, for example, Spectracel™ (available commercially from Sensient Technologies, Inc., St. Louis, Mo.), Benecel™ (available commercially from Ashland, Inc., Covington, Ky.), Methocel™ E15 Premium LV (available commercially from DOW Chemical Company, Midland, Mich.), among others.

In some embodiments, the amount of HPMC (by weight of the non-water components) in the composition may be at least about 40.0%, at least about 41.0%, at least about 42.0%, at least about 43.0%, at least about 44.0%, at least about 45.0%, at least about 46.0%, at least about 47.0%, at least about 48.0%, at least about 49.0%, at least about 50.0%, at least about 51.0%, at least about 52.0%, at least about 53.0%, at least about 54.0%, at least about 55.0%, at least about 56.0%, at least about 57.0%, at least about 58.0%, at least about 59.0%, at least about 60.0%, at least about 61.0%, at least about 62.0%, at least about 63.0%, at least about 64.0%, at least about 65.0%, at least about 66.0%, at least about 67.0%, at least about 68.0%, at least about 69.0%, at least about 70.0%, at least about 71.0%, at least about 72.0%, at least about 73.0%, at least about 74.0%, or at least about 75.0%. Further, the amount of HPMC (by weight of the non-water components) in the composition may be at most about 80.0%, at most about 79.0%, at most about 78.0%, at most about 77.0%, at most about 76.0%, at most about 75.0%, at most about 74.0%, at most about 73.0%, at most about 72.0%, at most about 71.0%, at most about 70.0%, at most about 69.0%, at most about 68.0%, at most about 67.0%, at most about 66.0%, at most about 65.0%, at most about 64.0%, at most about 63.0%, at most about 62.0%, at most about 61.0%, at most about 60.0%, at most about 59.0%, at most about 58.0%, at most about 57.0%, at most about 56.0%, at most about 55.0%, at most about 54.0%, at most about 53.0%, at most about 52.0%, at most about 51.0%, at most about 50.0%, at most about 49.0%, at most about 48.0%, at most about 47.0%, at most about 46.0%, or at most about 45.0%. This includes embodiments where the HPMC may be present in the composition in amounts (by weight of the non-water components) ranging from about 40.0% to about 80.0%, such as from about 50.0% to about 75.0%, or from about 55.0% to about 70.0%.

The HPMC may have a viscosity in 2% aqueous solution at 20° C. of at least about 1.0 cP, at least about 2.0 cP, at least about 3.0 cP, at least about 4.0 cP, at least about 5.0 cP, at least about 6.0 cP, at least about 7.0 cP, at least about 8.0 cP, at least about 9.0 cP, at least about 10.0 cP, at least about 11.0 cP, at least about 12.0 cP, at least about 13.0 cP, at least about 14.0 cP, or at least about 15.0 cP. The HPMC may have a viscosity in 2% aqueous solution at 20° C. of at most about 25.0 cP, at most about 24.0 cP, at most about 23.0 cP, at most about 22.0 cP, at most about 21.0 cP, at most about 20.0 cP, at most about 19.0 cP, at most about 18.0 cP, at most about 17.0 cP, at most about 16.0 cP, at most about 15.0 cP, at most about 14.0 cP, at most about 13.0 cP, at most about 12.0 cP, at most about 11.0 cP, at most about 10.0 cP, at most about 9.0 cP, at most about 8.0 cP, at most about 7.0 cP, at most about 6.0 cP, or at most about 5.0 cP. This includes embodiments where the HPMC may have a viscosity in 2% aqueous solution at 20° C. ranging from about 1.0 cP to about 25.0 cP, such as from about 5.0 cP to about 20.0 cP, or from about 12.0 cP to about 18.0 cP. In certain embodiments, the HPMC may have a viscosity in 2% aqueous solution at 20° C. of about 15.0 cP.

b. Triesters of Citric Acid

Triesters of citric acid used in edible products may be used to form the edible coating composition. Examples of triesters of citric acid include, without limitation, at least one of triethyl citrate, tripropyl citrate, tributyl citrate, acetyl triethyl citrate, acetyl tributyl citrate, and combinations thereof. Particularly suitable triesters of citric acid include, without limitation, triethyl citrate. Examples of commercially-available triesters of citric acid include triethyl citrate from Penta International Corp., Livingston, N.J., among others.

In some embodiments, the amount of triester of citric acid (by weight of the non-water components) in the composition may be at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.0%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.1%, at least about 3.2%, at least about 3.3%, at least about 3.4%, at least about 3.5%, at least about 3.6%, at least about 3.7%, at least about 3.8%, at least about 3.9%, at least about 4.0%, at least about 4.1%, at least about 4.2%, at least about 4.3%, at least about 4.4%, at least about 4.5%, at least about 4.6%, at least about 4.7%, at least about 4.8%, at least about 4.9%, at least about 5.0%, at least about 5.5%, at least about 6.0%, at least about 6.5%, at least about 7.0%, at least about 7.5%, at least about 8.0%, at least about 8.5%, at least about 9.0%, or at least about 9.5%. Further, the amount of triester of citric acid (by weight of the non-water components) in the composition may be at most about 10.0%, at most about 9.5%, at most about 9.0%, at most about 8.5%, at most about 8.0%, at most about 7.5%, at most about 7.4%, at most about 7.3%, at most about 7.2%, at most about 7.1%, at most about 7.0%, at most about 6.9%, at most about 6.8%, at most about 6.7%, at most about 6.6%, at most about 6.5%, at most about 6.4%, at most about 6.3%, at most about 6.2%, at most about 6.1%, at most about 6.0%, at most about 5.9%, at most about 5.8%, at most about 5.7%, at most about 5.6%, at most about 5.5%, at most about 5.4%, at most about 5.3%, at most about 5.2%, at most about 5.1%, at most about 5.0%, at most about 4.9%, at most about 4.8%, at most about 4.7%, at most about 4.6%, at most about 4.5%, at most about 4.4%, at most about 4.3%, at most about 4.2%, at most about 4.1%, at most about 4.0%, at most about 3.9%, at most about 3.8%, at most about 3.7%, at most about 3.6%, at most about 3.5%, at most about 3.4%, at most about 3.3%, at most about 3.2%, at most about 3.1%, at most about 3.0%, at most about 2.9%, at most about 2.8%, at most about 2.7%, at most about 2.6%, at most about 2.5%, at most about 2.4%, at most about 2.3%, at most about 2.2%, at most about 2.1%, at most about 2.0%, at most about 1.9%, at most about 1.8%, at most about 1.7%, at most about 1.6%, at most about 1.5%, at most about 1.4%, at most about 1.3%, at most about 1.2%, at most about 1.1%, at most about 1.0%, at most about 0.9%, at most about 0.8%, at most about 0.7%, at most about 0.6%, or at most about 0.5%. This includes embodiments where the triester of citric acid may be present in the composition in amounts (by weight of the non-water components) ranging from about 0.1% to about 10.0%, such as from about 0.5% to about 7.5%, from about 1.0% to about 6.0%, from about 4.0% to about 6.0%, from about 1.0% to about 3.0%, or from about 2.0% to about 5.0%.

c. Lecithin

Lecithin may be used to form the edible coating compositions.

In some embodiments, the lecithin may be selected from the group consisting of sunflower lecithin, soy lecithin, egg lecithin, peanut lecithin, sesame lecithin, canola lecithin, and combinations thereof. In certain embodiments, the lecithin may be sunflower lecithin. Examples of commercially-available lecithin include Topcithin™ (available commercially from Cargill, Inc., Minneapolis, Minn.), among others.

In some embodiments, the amount of lecithin (by weight of the non-water components) in the composition may be at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.0%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.1%, at least about 3.2%, at least about 3.3%, at least about 3.4%, at least about 3.5%, at least about 3.6%, at least about 3.7%, at least about 3.8%, at least about 3.9%, at least about 4.0%, at least about 4.1%, at least about 4.2%, at least about 4.3%, at least about 4.4%, at least about 4.5%, at least about 4.6%, at least about 4.7%, at least about 4.8%, at least about 4.9%, at least about 5.0%, at least about 5.1%, at least about 5.2%, at least about 5.3%, at least about 5.4%, at least about 5.5%, at least about 5.6%, at least about 5.7%, at least about 5.8%, at least about 5.9%, at least about 6.0%, at least about 6.1%, at least about 6.2%, at least about 6.3%, at least about 6.4%, at least about 6.5%, at least about 6.6%, at least about 6.7%, at least about 6.8%, at least about 6.9%, at least about 7.0%, at least about 7.1%, at least about 7.2%, at least about 7.3%, at least about 7.4%, at least about 7.5%, at least about 8.0%, at least about 8.5%, at least about 9.0%, at least about 9.5%, at least about 10.0%, at least about 10.5%, at least about 11.0%, at least about 11.5%, at least about 12.0%, at least about 12.5%, at least about 13.0%, at least about 13.5%, at least about 14.0%, or at least about 14.5%. In some embodiments, the amount of lecithin (by weight of the non-water components) in the composition may be at most about 15.0%, at most about 14.5%, at most about 14.0%, at most about 13.5%, at most about 13.0%, at most about 12.5%, at most about 12.0%, at most about 11.5%, at most about 11.0%, at most about 10.5%, at most about 10.0%, at most about 9.5%, at most about 9.0%, at most about 8.5%, at most about 8.0%, at most about 7.5%, at most about 7.4%, at most about 7.3%, at most about 7.2%, at most about 7.1%, at most about 7.0%, at most about 6.9%, at most about 6.8%, at most about 6.7%, at most about 6.6%, at most about 6.5%, at most about 6.4%, at most about 6.3%, at most about 6.2%, at most about 6.1%, at most about 6.0%, at most about 5.9%, at most about 5.8%, at most about 5.7%, at most about 5.6%, at most about 5.5%, at most about 5.4%, at most about 5.3%, at most about 5.2%, at most about 5.1%, at most about 5.0%, at most about 4.9%, at most about 4.8%, at most about 4.7%, at most about 4.6%, at most about 4.5%, at most about 4.4%, at most about 4.3%, at most about 4.2%, at most about 4.1%, at most about 4.0%, at most about 3.9%, at most about 3.8%, at most about 3.7%, at most about 3.6%, at most about 3.5%, at most about 3.4%, at most about 3.3%, at most about 3.2%, at most about 3.1%, at most about 3.0%, at most about 2.9%, at most about 2.8%, at most about 2.7%, at most about 2.6%, at most about 2.5%, at most about 2.4%, at most about 2.3%, at most about 2.2%, at most about 2.1%, at most about 2.0%, at most about 1.9%, at most about 1.8%, at most about 1.7%, at most about 1.6%, at most about 1.5%, at most about 1.4%, at most about 1.3%, at most about 1.2%, at most about 1.1%, or at most about 1.0%. This includes embodiments where the lecithin may be present in the composition in an amount (by weight of the non-water components) ranging from about 0.1% to about 15.0%, such as from about 0.5% to about 10.0%, or from about 1.0% to about 5.0%.

d. C₁₂₋₂₄ Fatty Acids

C₁₂₋₂₄ fatty acids may be used to form the edible coating compositions. Examples of suitable C₁₂₋₂₄ fatty acids include, without limitation, stearic acid, oleic acid, palmitic acid, arachidic acid, myristic acid, and combinations thereof. Particularly suitable C₁₂₋₂₄ fatty acids include, without limitation, stearic acid. Examples of commercially-available stearic acid includes Tristar NF (available commercially from American International Chemical, Inc., Framingham, Mass.), among others.

In some embodiments, the C₁₂₋₂₄ fatty acid may be saturated or unsaturated.

In some embodiments, the amount of C₁₂₋₂₄ fatty acid (by weight of the non-water components) of the composition may be at least about 10.0%, at least about 11.0%, at least about 12.0%, at least about 13.0%, at least about 14.0%, at least about 15.0%, at least about 16.0%, at least about 17.0%, at least about 18.0%, at least about 19.0%, at least about 20.0%, at least about 21.0%, at least about 22.0%, at least about 23.0%, at least about 24.0%, at least about 25.0%, at least about 26.0%, at least about 27.0%, at least about 28.0%, at least about 29.0%, 30.0%, at least about 31.0%, at least about 32.0%, at least about 33.0%, at least about 34.0%, at least about 35.0%, at least about 36.0%, at least about 37.0%, at least about 38.0%, at least about 39.0%, at least about 40.0%, at least about 41.0%, at least about 42.0%, at least about 43.0%, at least about 44.0%, at least about 45.0%, at least about 46.0%, at least about 47.0%, at least about 48.0%, or at least about 49.0%. Further, the amount of C₁₂₋₂₄ fatty acid (by weight of the non-water components) in the composition may be at most about 50.0%, at most about 49.0%, at most about 48.0%, at most about 47.0%, at most about 46.0%, at most about 45.0%, at most about 44.0%, at most about 43.0%, at most about 42.0%, at most about 41.0%, at most about 40.0%, at most about 39.0%, at most about 38.0%, at most about 37.0%, at most about 36.0%, at most about 35.0%, at most about 34.0%, at most about 33.0%, at most about 32.0%, at most about 31.0%, at most about 30.0%, at most about 29.0%, at most about 28.0%, at most about 27.0%, at most about 26.0%, at most about 25.0%, at most about 24.0%, at most about 23.0%, at most about 22.0%, at most about 21.0%, at most about 20.0%, at most about 19.0%, at most about 18.0%, at most about 17.0%, at most about 16.0%, at most about 15.0%, at most about 14.0%, at most about 13.0%, at most about 12.0%, at most about 11.0%. This includes embodiments where the C₁₂₋₂₄ fatty acid may be present in the composition in amount (by weight of the non-water components) ranging from about 10.0% to about 50.0%, such as from about 15.0% to about 45.0%, from about 15.0% to about 35.0%, from about 20.0% to about 40.0%, or from about 25.0% to about 35.0%.

In some embodiments, the C₁₂₋₂₄ fatty acid may have a small average particle size prior to addition to the composition. In some embodiments, the C₁₂₋₂₄ fatty acid may have an average particle size of not more than (NMT) about 500 microns, NMT about 450 microns, NMT about 400 microns, NMT about 350 microns, NMT about 300 microns, NMT about 250 microns, NMT about 200 microns, NMT about 190 microns, NMT about 180 microns, NMT about 170 microns, NMT about 160 microns, NMT about 150 microns, NMT about 140 microns, NMT about 130 microns, NMT about 120 microns, NMT about 110 microns, NMT about 100 microns, NMT about 95 microns, NMT about 90 microns, NMT about 85 microns, NMT about 80 microns, NMT about 75 microns, NMT about 70 microns, NMT about 65 microns, NMT about 60 microns, NMT about 55 microns, NMT about 50 microns, NMT about 45 microns, NMT about 40 microns, NMT about 35 microns, NMT about 30 microns, NMT about 25 microns, NMT about 20 microns, NMT about 15 microns, or NMT about 10 microns. In some embodiments, 98% of the C₁₂₋₂₄ fatty acid passes through a mesh size of at most about 1000 mesh, at most about 900 mesh, at most about 800 mesh, at most about 700 mesh, at most about 600 mesh, at most about 500 mesh, at most about 450 mesh, at most about 400 mesh, at most about 350 mesh, at most about 300 mesh, at most about 250 mesh, at most about 200 mesh, at most about 190 mesh, at most about 180 mesh, at most about 170 mesh, at most about 160 mesh, at most about 150 mesh, at most about 140 mesh, at most about 130 mesh, at most about 120 mesh, at most about 110 mesh, at most about 100 mesh, at most about 95 mesh, at most about 90 mesh, at most about 85 mesh, at most about 80 mesh, at most about 75 mesh, at most about 70 mesh, at most about 65 mesh, at most about 60 mesh, at most about 55 mesh, at most about 50 mesh, at most about 45 mesh, at most about 40 mesh, at most about 35 mesh, at most about 30 mesh, at most about 25 mesh, at most about 20 mesh, at most about 15 mesh, or at most about 10 mesh.

In some embodiments, the edible coating composition may comprise a ratio (by weight) of HPMC to C₁₂₋₂₄ fatty acid of at least about 1.0:1, at least about 1.1:1, at least about 1.2:1, at least about 1.3:1, at least about 1.4:1, at least about 1.5:1, at least about 1.6:1, at least about 1.7:1, at least about 1.8:1, at least about 1.9:1, at least about 2.0:1, at least about 2.1:1, at least about 2.2:1, at least about 2.3:1, at least about 2.4:1, at least about 2.5:1, at least about 2.6:1, at least about 2.7:1, at least about 2.8:1, at least about 2.9:1, at least about 3.0:1, at least about 3.1:1, at least about 3.2:1, at least about 3.3:1, at least about 3.4:1, at least about 3.5:1, at least about 3.6:1, at least about 3.7:1, at least about 3.8:1, at least about 3.9:1, at least about 4.0:1, at least about 4.1:1, at least about 4.2:1, at least about 4.3:1, at least about 4.4:1, at least about 4.5:1, at least about 4.6:1, at least about 4.7:1, at least about 4.8:1, or at least about 4.9:1. Further, the compositions may comprise a ratio (by weight) of HPMC to C₁₂₋₂₄ fatty acid of at most about 5.0:1, at most about 4.9:1, at most about 4.8:1, at most about 4.7:1, at most about 4.6:1, at most about 4.5:1, at most about 4.4:1, at most about 4.3:1, at most about 4.2:1, at most about 4.1:1, at most about 4.0:1, at most about 3.9:1, at most about 3.8:1, at most about 3.7:1, at most about 3.6:1, at most about 3.5:1, at most about 3.4:1, at most about 3.3:1, at most about 3.2:1, at most about 3.1:1, at most about 3.0:1, at most about 2.9:1, at most about 2.8:1, at most about 2.7:1, at most about 2.6:1, at most about 2.5:1, at most about 2.4:1, at most about 2.3:1, at most about 2.2:1, at most about 2.1:1, at most about 2.0:1, at most about 1.9:1, at most about 1.8:1, at most about 1.7:1, at most about 1.6:1, at most about 1.5:1, at most about 1.4:1, at most about 1.3:1, at most about 1.2:1, or at most about 1.1:1. This includes embodiments where the composition may have a ratio (by weight) of HPMC to C₁₂₋₂₄ fatty acid ranging from about 1:1 to about 5:1, such as from about 1.5:1 to about 4:1, or from about 2:1 to about 3:1.

In some embodiments, the edible coating compositions may comprise a ratio (by weight) of C₁₂₋₂₄ fatty acid to triester of citric acid of at least about 1:1, at least about 2:1, at least about 3:1, at least about 4:1, at least about 5:1, at least about 5.5:1, at least about 6:1, at least about 6.5:1, at least about 7:1, at least about 7.5:1, at least about 8:1, at least about 8.5:1, at least about 9:1, at least about 9.5:1, at least about 10:1, at least about 10.5:1, at least about 11:1, at least about 11.5:1, at least about 12:1, at least about 12.5:1, at least about 13:1, at least about 13.5:1, at least about 14:1, at least about 14.5:1, at least about 15:1, at least about 15.5:1, at least about 16:1, at least about 16.5:1, at least about 17:1, at least about 17.5:1, at least about 18:1, at least about 18.5:1, or at least about 19:1, at least about 19.5:1, at least about 20:1, at least about 21:1, at least about 22:1, at least about 23:1, at least about 24:1, at least about 25:1, at least about 26:1, at least about 27:1, at least about 28:1, at least about 29:1, at least about 30:1, at least about 31:1, at least about 32:1, at least about 33:1, at least about 34:1, at least about 35:1, at least about 36:1, at least about 37:1, at least about 38:1, at least about 39:1, or at least about 40:1. Further, the compositions may comprise a ration (by weight) of C₁₂₋₂₄ fatty acid to triester of citric acid of at most about 40:1, at most about 39:1, at most about 38:1, at most about 37:1, at most about 36:1, at most about 35:1, at most about 34:1, at most about 33:1, at most about 32:1, at most about 31:1, at most about 30:1, at most about 29:1, at most about 28:1, at most about 27:1, at most about 26:1, at most about 25:1, at most about 24:1, at most about 23:1, at most about 22:1, at most about 21:1, at most about 20:1, at most about 19.5:1, at most about 19:1, at most about 18.5:1, at most about 18:1, at most about 17.5:1, at most about 17:1, at most about 16.5:1, at most about 16:1, at most about 15.5:1, at most about 15:1, at most about 14.5:1, at most about 14:1, at most about 13.5:1, at most about 13:1, at most about 12.5:1, at most about 12:1, at most about 11.5:1, at most about 11:1, at most about 10.5:1, at most about 10:1, at most about 9.5:1, at most about 9:1, at most about 8.5:1, at most about 8:1, at most about 7.5:1, at most about 7:1, at most about 6.5:1, at most about 6:1, at most about 5.5:1, at most about 5:1, at most about 4:1, at most about 3:1, or at most about 2:1. This includes embodiments where the composition may have a ratio (by weight) of C₁₂₋₂₄ fatty acid to triester of citric acid ranging from about 1:1 to about 40:1, such as from about 2:1 to about 30:1, or from about 5:1 to about 20:1.

e. Opacifying Agent

The edible coating compositions disclosed herein may optionally contain an opacifying agent. Opacifying agents include, for example, titanium dioxide, clays, and divalent salts such as zinc oxides, dicalcium phosphate, dicalcium phosphate dehydrate, tricalcium phosphate, calcium carbonate, and precipitated calcium carbonate. In certain embodiments, the opacifying agent may comprise titanium dioxide. Examples of commercial sources of titanium dioxide include, but are not limited to, Brenntag Specialties, Inc., Germany; Kronos Canada, Inc., Canada; Huntsman Pigments, The Woodlands, Tex.; and the like.

In some embodiments, the amount of opacifying agent (by weight of the non-water components) in the composition may be at least about 0.1%, at least about 0.5%, at least about 1.0%, at least about 1.5%, at least about 2.0%, at least about 2.5%, at least about 3.0%, at least about 3.5%, at least about 4.0%, at least about 4.5%, at least about 5.0%, at least about 6.0%, at least about 7.0%, at least about 8.0%, at least about 9.0%, at least about 10.0%, at least about 11.0%, at least about 12.0%, at least about 13.0%, at least about 14.0%, at least about 15.0%, at least about 16.0%, at least about 17.0%, at least about 18.0%, at least about 19.0%, at least about 20.0%, at least about 21.0%, at least about 22.0%, at least about 23.0%, at least about 24.0%, or at least about 25.0%. In some embodiments, the amount of opacifying agent (by weight of the non-water components) in the composition may be at most about 30.0%, at most about 29.0%, at most about 28.0%, at most about 27.0%, at most about 26.0%, at most about 25.0%, at most about 24.0%, at most about 23.0%, at most about 22.0%, at most about 21.0%, at most about 20.0%, at most about 19.0%, at most about 18.0%, at most about 17.0%, at most about 16.0%, at most about 15.0%, at most about 14.0%, at most about 13.0%, at most about 12.0%, at most about 11.0%, at most about 10.0%, at most about 9.0%, at most about 8.0%, at most about 7.0%, at most about 6.0%, at most about 5.0%, at most about 4.0%, at most about 3.0%, at most about 2.0, or at most about 1.0%. This includes embodiments where the opacifying agent may be present in the composition in amounts (by weight of the non-water components) ranging from about 0.1% to about 30.0%, such as from about 5.0% to about 20.0%, or from about 10.0% to about 25.0%.

f. Water

The edible coating compositions disclosed herein may optionally contain water. In some embodiments containing water, the amount of water (by weight of the entire composition) in the composition may be at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. Further, in some embodiments containing water, the amount of water (by weight of the entire composition) in the composition may be at most about 99.9%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, or at most about 5%. This includes embodiments where the water may be present in the composition in amount (by weight of the entire composition) ranging from about 1% to about 99.9%, such as from about 5% to about 95%, or from about 50% to about 99%. In some embodiments, the balance of the edible coating composition may be water.

g. Additional Composition Additives

The edible coating compositions disclosed herein may also comprise additional additives known to those of skill in the art. Examples of suitable additives include, without limitation, colorants (synthetic, natural exempt and natural non-exempt), flavorings, sweeteners, sodium bicarbonate, sodium stearate, and the like. In some embodiments, additional additives do not affect at least one of the moisture uptake, stability, or the clarity of the edible coatings.

Examples of colorants include dyes, lakes, and pigments and may include, but are not limited to, titanium dioxide, iron oxides, dyes such as, for example, FD&C Lakes, Carmine Lake, FD&C Blue no. 1, FD&C Blue no. 2, FD&C Red no. 3, FD&C Red no. 40, FD&C Yellow no. 5, FD&C Yellow no. 6, FD&C Green no. 3, alumina, talc, annatto extract, calcium carbonate, canthaxanthin, caramel, β-carotene, carmine, dihydroxyacetone, turmeric oleoresin, cochineal extract, gardenia yellow, gardenia blue, beet powder, grape skin extract, riboflavin, purple sweet potato, red sweet potato, chlorophyll-containing extracts, purple blend (available from Sensient Colors, Inc., No. 53219), carmine high tint, pearlescent pigments, SensiPearl™, Intense Silver, and Bright Silver (available from Sensient Colors, Inc.), natural colorants, and the like. Other examples of colorants are found in 21 C.F.R. §§73 and 74, which are hereby fully incorporated by reference.

Examples of flavorings may be synthetic or artificial flavorings, natural flavorings or any mixture thereof and may include, but are not limited to, sensates, flavonoids, antioxidants, natural flavorants, synthetic flavorants, bioflavonoids, flavones, flavone, flavonol, flavanonol, isoflavones, ethyl vanillin, tangerine flavor, lemon flavor, lemon extract, liquid caramel, spearmint oil, orange flavor, almond, amaretto, apple, green apple, apple-cherry-berry, apple-honey, apricot, bacon, balls of fire, banana, barbeque, beef, roast beef, beef steak, berry, berry blue, birch beer/spruce beer, blackberry, bloody mary, blueberry, boysenberry, brandy, bubble gum, butter, butter pecan, buttermilk, butterscotch, candy corn, cantaloupe, cantaloupe lime, caramel, carrot, cassia, caviar, celery, cereal, champagne, cherry, cherry cola, cherry maraschino, wild cherry, black cherry, red cherry, cherry-cola, chicken, chocolate, chocolate almond, cinnamon spice, citrus, citrus blend, citrus-strawberry, clam, cocoa, coconut, toasted coconut, coffee, coffee almond, cola, cola-vanilla, cookies & cream, cool, cotton candy, cranberry, cranberry-raspberry, cream, cream soda, dairy type cream, crème de menthe, cucumber, black currant, dulce de leche, egg nog, pork fat, type fat, anchovy fish, herring fish, sardine fish, frankfurter, fiery hot, fried garlic, sauteed garlic, gin, ginger ale, ginger beer, graham cracker type, grape, grape grapefruit, grapefruit-lemon, grapefruit-lime, grenadine, grill, guarana, guava, hazelnut, honey, hot, roasted honey, ice cream cone, jalapeno, key lime, kiwi, kiwi-banana, kiwi-lemon-lime, kiwi-strawberry, kola champagne, lard type, lemon, lemon custard, lemonade, pink lemonade, lemon-lime, lime, malt, malted milk, mango, mango-pineapple, maple, margarita, marshmallow, meat type, condensed milk, cooked milk, mint, mirepoix, mocha, mochacinna, molasses, mushroom, sauteed mushroom, muskmelon, nectarine, neapolitan, green onion, sautéed onion, orange, orange cordial, orange creamsicle, orange creme, orange peach mango, orange strawberry banana, creamy orange, mandarin orange, orange-passion-guava, orange-pineapple, papaya, passion fruit, peach, peach mango, peanut, roasted peanut, pear, pecan danish type, pecan praline, pepper, peppermint, pimento, pina colada, pina colada/pineapple-coconut, pineapple, pineapple-orange, pistachio, pizza, pomegranate, pork fat type, baked potato, prune, punch, citrus punch, tropical punch, cherry fruit punch, grape punch, raspberry, black raspberry, blue raspberry, red raspberry, raspberry-blackberry, raspberry-ginger ale, raspberry-lime, roast type, root beer, rum, sangria, sarsaparilla, sassafras, sausage, sausage pizza, savory, seafood, shrimp, hickory smoke, mesquite smoke, sour, sour cream, sour cream and onion, spearmint, spicy, strawberry, strawberry margarita, jam type strawberry, strawberry-kiwi, burnt sugar, sweet & sour, tallow, tamarind, tangerine-lime, tangerine, tea, tequila type, toffee, triple sec, tropical fruit mix, turkey, tutti frutti, vanilla, vanilla cream, vanilla custard, french vanilla, vegetable, vermouth, vinegar, balsamic vinegar, watermelon, whiskey, wildberry, wine, and yogurt, and the like. Other examples of flavors are found in 21 C.F.R. .sctn..sctn.172.510, 172.515, 172.520, 172.530, 172.535, 172.575, 172.580 and 172.585, which are hereby fully incorporated by reference. A variety of food grade flavors are commercially-available from Sensient Flavors Inc. in Indianapolis, Ind., Givaudan SA in Cincinnati, Ohio, and International Flavors & Fragrance in New York, N.Y.

In some embodiments, the edible coating has a solids content that enhances the uniformity of the coating. In some embodiments, the edible coating comprises a solids content that enhances the uniformity of the coating. In some embodiments, the edible coating composition may have a solids content of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the edible coating composition may have a solids content of at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, or at most about 1%. This includes embodiments where the edible coating composition may have solids contents ranging from about 0.1% to about 100%, such as solids contents ranging from about 0.5% to about 20.0%, or solids contents ranging from about 1.0% to about 15.0%.

The edible coating composition may have a viscosity of at least about 50 cP, at least about 100 cP, at least about 150 cP, at least about 200 cP, at least about 225 cP, at least about 250 cP, or at least about 275 cP. The edible coating composition may have a viscosity of at most at most about 400 cP, at most about 350 cP, at most about 335 cP, at most about 325 cP, at most about 300 cP, at most about 200 cP, or at most about 100 cP. This includes embodiments where the edible coating composition may have viscosities ranging from about 200 cP to about 400 cP, such as viscosities ranging from about 225 cP to about 375 cP, or viscosities ranging from about 250 cP to about 300 cP.

Viscosity of the composition may be assessed by any suitable method such as, for example, using a Brookfield viscometer equipped with a UL Adapter Assembly and UL Spindle (available from Brookfield Engineering Labs., Inc.). The edible coating composition or other test sample is poured into a clean graduated cylinder or other sample container, and the spindle is immersed in the sample. After starting the viscometer motor, the speed is adjusted to achieve a percent torque between 50-70%, and the viscosity (cP) of the sample is recorded.

### 2. Edible Coatings

Edible coatings may be formed by applying the edible coating compositions to a substrate. The edible coatings disclosed herein may comprise the components of the edible coating compositions minus any components that are removed in the process of preparing the edible film coating. The edible coatings disclosed herein may be formed on any suitable substrate.

In some embodiments, the edible coatings may comprise the components of the edible coating compositions minus any components that evaporate in the process of preparing the edible film coating. In some embodiments, the edible coatings may comprise the non-water components of the edible coating composition.

In some embodiments, the edible coatings may have reduced moisture uptake in a humid environment compared to conventional HPMC coatings. Moisture uptake is a measure of the amount of water taken up as measured by weight gain of a sample that has been placed in a controlled environment of a set humidity. Moisture uptake can be measured by any suitable method known to one of skill in the art. Examples of procedures for measuring moisture uptake include, but are not limited to, exposing a coated substrate to 60% relative humidity at 25° C. (“storage condition H2”) or 75% relative humidity at 40° C. (“storage condition H4”), or 40-60% relative humidity at 20° C. (room conditions) and comparing the initial mass or weight of the sample to the mass or weight at various time intervals, such as, for example, 5 minutes, 30 minutes, 1 hour, 2 hours, 6 hours, 12 hours, 18 hours, 24 hours, 36 hours, 48 hours, 72 hours, 5 days, 7 days, 10 days, 14 days, 21 days, 25 days, 1 month, 2 months, 3 months, 6 months, 1 year, and the like. Stable mass or weight indicates a low moisture uptake. Without wishing to be limited by any particular theory, it is believe that the physical dimensions of an edible coating increase at a rate that corresponds to the moisture uptake. Accordingly, the moisture uptake can be investigated by measuring the physical size of the edible coating at various time intervals, as indicated above.

In some embodiments, a substrate may be coated with an edible coating composition to form an edible coating at a 1% weight gain, a 2% weight gain, a 3% weight gain, a 4% weight gain, a 5% weight gain, a 6% weight gain, a 7% weight gain, a 8% weight gain, a 9% weight gain, or a 10% weight gain compared to the uncoated substrate. When exposed to storage condition H2, H4, or room conditions for a period of time equaling or exceeding 5 minutes, 30 minutes, 60 minutes, 90 minutes, 1 hour, 2 hours, 6 hours, 12 hours, 18 hours, 24 hours, 36 hours, 48 hours, 72 hours, 5 days, 7 days, 10 days, 14 days, 21 days, 25 days, 1 month, 2 months, 3 months, 6 months, 1 year, and the like as measured by a VTI-SA Sorption Analyzer (available commercially from TA Instruments, New Castle, Del.), the coated substrate or edible coating may exhibit a moisture uptake of at most about 4.0%, at most about 3.0%, at most about 2.5%, at most about 2.0%, at most about 1.9%, at most about 1.8%, at most about 1.7%, at most about 1.6%, at most about 1.5%, at most about 1.4%, at most about 1.3%, at most about 1.2%, at most about 1.1%, at most about 1.0%, at most about 0.95%, at most about 0.90%, at most about 0.85%, at most about 0.80%, at most about 0.75%, at most about 0.70%, at most about 0.65%, at most about 0.60%, at most about 0.55%, at most about 0.50%, at most about 0.45%, at most about 0.40%, at most about 0.35%, at most about 0.30%, at most about 0.25%, at most about 0.20%, at most about 0.15%, at most about 0.10% or at most 0.001%.

In some embodiments, the coated substrate or edible coating may have a moisture uptake, as measured by weight gain over time after exposure to storage condition H2, H4 or room conditions for the above-mentioned time durations, of at most about 0.1 grams per hour (g/hr), at most about 0.05 g/hr, at most about 0.04 g/hr, at most about 0.03 g/hr, at most about 0.02 g/hr, at most about 0.015 g/hr, at most about 0.01 g/hr, at most about 0.009 g/hr, at most about 0.008 g/hr, at most about 0.007 g/hr, at most about 0.006 g/hr, at most about 0.005 g/hr, at most about 0.004 g/hr, at most about 0.003 g/hr, at most about 0.002 g/hr, at most about 0.001 g/hr, or at most about 0.0005 g/hr. In some embodiments, the coated substrate or edible coating may have a moisture uptake, as measured by weight gain over time after exposure to storage condition H2, H4 or room conditions for the above-mentioned time durations, of at most about 0.01 inches per hour (in/hr), at most about 0.005 in/hr, at most about 0.004 in/hr, at most about 0.003 in/hr, at most about 0.002 in/hr, at most about 0.0015 in/hr, at most about 0.001 in/hr, at most about 0.0009 in/hr, at most about 0.0008 in/hr, at most about 0.0007 in/hr, at most about 0.0006 in/hr, at most about 0.0005 in/hr, at most about 0.0004 in/hr, at most about 0.0003 in/hr, at most about 0.0002 in/hr, at most about 0.0001 in/hr, or at most about 0.00005 in/hr.

In some embodiments, the edible coatings may have improved slip properties. Slip is a measure of the friction between surfaces, generally surfaces of coated dosage units. Improved slip indicates that less force is required. Additionally, improved slip properties may improve the swallowability and subject compliance of the edible coatings. Swallowability testing was performed by Sensient's Sensory Testing Laboratories at Hoffman Estates in Illinois. The coatings can be tested based on the following: overall appearance, color/glossiness, texture/feel, mouth feel—taste perception, mouth feel—swallowability, slip and mouth feel—swallowability, tablet shape and feel. Additionally, swallowability may be examined by radioactivity, as well as “synthetic esophagus” methods.

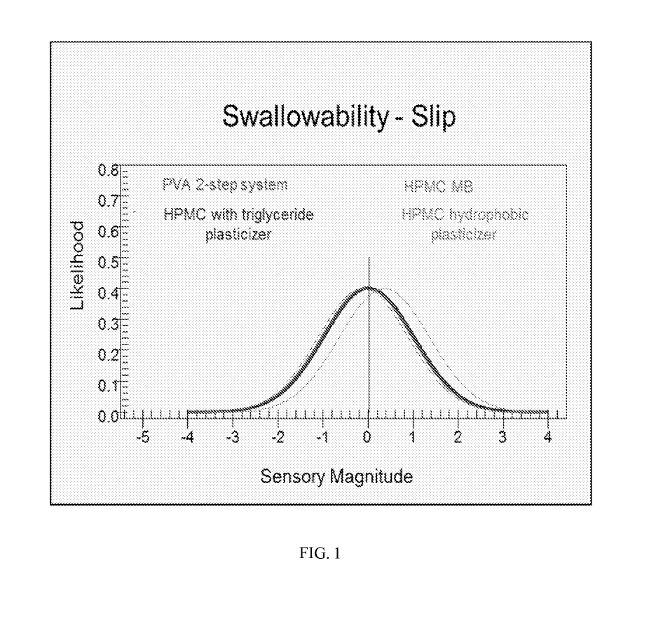

The four coatings tested were, as listed, a two-step coating system where a base coat was applied, and a top coating applied thereafter, an HPMC coating system with triglycerides, an HPMC moisture barrier as noted herein, and a HPMC hygroscopic plasticizer. The slip was tested and was comparatively different with higher slip with the formulation mentioned herein as seen in the attached Swallowability Slip graph (FIG. 1).

Slip can be measured by any suitable method known to one of skill in the art. Examples of procedures for measuring slip include, but are not limited to, smoothness measurements, among others.

Smoothness of the edible coatings may be measured by utilizing a TA.XT.Plus Texture Analyzer with a vertical friction rig serving as a force arm. The probe height and force may be calibrated and the return force arm positioned 1 mm from the bottom of the container. A non-friction gliding object, such as a piece of paper tissue, may be attached to the vertical probe of the rig using heavy-duty double-sided tape. A tablet or other substrate coated with an edible coating may be attached to the vertical wall of the rig using heavy-duty double-sided tape. A weight (e.g., 100 g) is added to the horizontal plate of the probe, and incremental increases of force are applied to the non-friction glidant in contact with the edible coating until the glidant slips or moves to measure the tension force. A smooth edible coating may exhibit a slip at a tension of at least about 10 g, at least about 20 g, and at least about 30 g tension force. A smooth edible coating may exhibit a slip at a tension of less than about 100 g, less than about 90 g, less than about 80 g, less than about 70 g, less than about 60 g, less than about 50 g, less than about 40 g, less than about 30 g, and less than about 20 g friction force. A smooth edible coating may exhibit a slip at a friction force from about 10 g to about 100 g, a friction force from about 20 g to about 70 g, a friction force from about 30 g to about 50 g, a friction force from about 35 g to about 45 g, or a friction force from about 35 g to about 40 g.

In some embodiments, the coatings may exhibit high hardness. Hardness can be measured by any suitable method known to one of skill in the art. Examples of procedures for measuring hardness include, but are not limited to, Dr. Schleuniger Pharmatron 6D tablet tester using testing method set at kilopond (kp) force. Coatings disclosed herein may be capable of increasing the hardness of softer substrates upon coating or maintaining the hardness of harder substrates upon coating. In some embodiments, the coatings may have a hardness of at least about 10 kp, at least about 11 kp, at least about 12 kp, at least about 13 kp, at least about 14 kp, at least about 15 kp, at least about 16 kp, at least about 17 kp, at least about 18 kp, at least about 19 kp, at least about 20 kp, at least about 21 kp, at least about 22 kp, at least about 23 kp, at least about 24 kp, at least about 25 kp, at least about 26 kp, at least about 27 kp, at least about 28 kp, at least about 29 kp, at least about 30 kp, at least about 31 kp, at least about 32 kp, at least about 33 kp, at least about 34 kp, at least about 35 kp, at least about 36 kp, at least about 37 kp, at least about 38 kp, at least about 39 kp, or at least about 40 kp. This includes a hardness of about 10 kp to about 40 kp, a hardness of about 15 kp to about 35 kp, or a hardness of about 20 kp to about 30 kp.

In some embodiments, the coatings may exhibit an immediate release profile. Examples of procedures for determining the release profile include, but are not limited to, immediate release disintegration tests, such as USP32/NF27 S2, Chapter 2040 (Disintegration and Dissolution of Dietary Supplements), Chapter 701 (Disintegration) testing criteria, and the like. In some embodiments, the coatings may provide for release (e.g., of contents contained therein) within a particular time period. The time period may be no longer than 120 minutes, 90 minutes, 60 minutes, 45 minutes, 30 minutes, 25 minutes, 20 minutes, 15 minutes, 10 minutes, 5 minutes, 3 minutes or 2 minutes.

In some embodiments, the coatings may exhibit good adhesion to a substrate. Adhesion is a measure of how strongly the coating binds to the substrate. Adhesion of the coatings may be measured using a TA.XT.Plus Texture Analyzer (available from Texture Technologies Corp.) equipped with a 25 mm stainless steel cylindrical probe. A substrate coated with an enteric coating is scored around its hemisphere with a sharp blade and then attached to the top of the flat platform of the texture analyzer using heavy-duty double-sided tape (available from 3M). Another piece of heavy-duty double-sided tape is pressed to the bottom of the cylindrical probe, and the probe is then compressed to 800 g force onto the coated substrate for 10 seconds. The probe is then pulled away from the coated substrate at a rate of 1 mm/second, measuring the tension force until either (1) the coating separates from the substrate, or (2) the tape separates from the coating. Where the coating remains on the substrate, the measured force equals the force required to pull the double-sided tape away from the coating, and the adhesion force of the coating to the substrate is thus greater than the measured adhesion force between the tape and the coating. An adherent coating may exhibit an adhesion force between the coating and the substrate of at least about 50 g force, at least about 100 g force, at least about 200 g force, at least about 400 g force, at least about 600 g force, at least about 700 g force, at least about 800 g force, at least about 900 g force, or at least about 1000 g force.

In some embodiments, the coatings may exhibit low friability. Friability can be measured by any suitable method known to one of skill in the art. Examples of procedures for measuring friability include, but are not limited to, Dr. Schleuniger Pharmatron method FTV-2, U.S. Pharmacopeia Chapter 1216 Tablet Friability, measuring the weight difference after exposing tablets to rotating drums at 25 RPM±1 RPM for 100 revolutions, and the like. In some embodiments, the friability may be at most about 1%, at most about 0.1%, or at most about 0.01%.

In some embodiments, the coatings may exhibit good clarity. Examples of procedures for measuring clarity include, but are not limited to, measuring the lightness, whiteness or yellowness of an uncoated sample and comparing to the respective lightness, whiteness or yellowness of a coated sample. Smaller changes correspond to higher clarity. Lightness can be assessed by any suitable method such as, for example, measuring the Lightness Index according to ASTM Method E313 with a D65/10° illumination source (referred to as “E313 [D65/10]”) using a LabScan™ XE spectrophotometer (available from HunterLab, Inc.). A sample is loaded into the instrument's sample port and scanned, and the Lightness Index (E313 [D65/10]) value is calculated using measurements taken on the CIE L*a*b* color scale. The resulting Lightness Index value is used to assess lightness of the sample. Whiteness can be assessed by any suitable method, such as, for example, measuring the Whiteness Index according to ASTM Method E313 with a D65/10° illumination source (referred to as “WI E313 [D65/10]”) using a LabScan™ XE spectrophotometer (available from HunterLab, Inc.). A sample substrate coated with a film coating is loaded into the instrument's sample port and scanned, and the Whiteness Index (WI E313 [D65/10]) value is calculated using measurements taken on the CIE L*a*b* color scale. The resulting Whiteness Index value is used to assess whiteness of the sample. Yellowness can be assessed by any suitable method, such as, for example, measuring the Yellowness Index according to ASTM Method E313 with a D65/10° illumination source (referred to as “YI E313 [D65/10]”) using a LabScan™ XE spectrophotometer. A sample substrate coated with a film coating is loaded into the instrument's sample port and scanned, and the resulting YI E313 [D65/10] value is used to assess yellowness of the sample. In some embodiments, the change in lightness, whiteness or yellowness may be less than about 50%, less than about 25%, less than about 20%, less than about 15%, less than about 10%, less than about 5%, or less than about 1%.

In some embodiments, the coatings may have high whiteness and/or high opacity. Whiteness can be assessed as described above. Opacity can be assessed by evaluating the Whiteness Index achieved upon coating one or more colored substrate cores with edible coatings as described herein. For example, when coating a colored substrate, the Whiteness Index will increase with additional weight gain until high and/or full opacity is reached. Upon reaching high and/or full opacity, the Whiteness Index may level off relative to further increases in weight gain. Suitably, the disclosed film coatings may provide high and/or full opacity at a weight gain of less than about 10%, less than about 9%, less than about 8%, less than about 7%, less than about 6%, less than about 5%, less than about 4%, less than about 3%, and less than about 2% weight gain. For example, the disclosed edible coatings may provide high and/or full opacity at weight gains ranging from about 1% to about 10%, such as weight gains ranging from about 3% to about 9%, and weight gains ranging from about 2% to about 6%. In some embodiments, the disclosed film coating compositions, film coating suspensions, and/or film coatings comprise an opacifying agent in amounts sufficient to provide a film coating with high whiteness and/or full/high opacity at low weight gain.

In some embodiments, applying the composition to an edible substrate produces a coated edible substrate, the edible substrate and the coated edible substrate each having a gloss, wherein the gloss of the coated edible substrate may be from about 0% to about 500% greater than the gloss of the edible substrate. In some embodiments, the gloss of the coated edible substrate may be at least about 5% greater, at least about 10% greater, at least about 50% greater, at least about 100% greater, at least about 150% greater, at least about 200% greater, at least about 300% greater, at least about 400, or at least about 500% greater.

In some embodiments, applying the composition to an edible substrate produces a coated edible substrate, the edible substrate and the coated edible substrate each having a reflectance spectrum having a peak wavelength, a peak width and an integrated area, wherein the peak wavelength, peak width or integrated area may vary by less than about 50% from edible substrate to coated edible substrate. In some embodiments, the peak wavelength, peak width or integrated area may vary by less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, or less than about 0.1%.

In some embodiments, applying the composition to an edible substrate produces an edible coating on the edible substrate, the edible coating having a transmission haze of less than about 50% as measured by ASTM D1003-95, “Standard Test Method for Haze and Luminous Transmittance of Transparent Plastics” or a reflection haze of less than about 50% as measured by ASTM E430-91, “Standard Test Methods for Measurement of Gloss of High-Gloss Surfaces by Goniophotometry.” Gloss may be measured using Novocure equipment (Rhopoint Instruments, UK).

In some embodiments, the coatings may exhibit good stability. Stability indicates the ability for a coating to remain functional under normal or abnormal storage conditions. One example of a means of testing stability is exposing a coated substrate to 60% relative humidity at 25° C. (“storage condition H2”), 75% relative humidity at 40° C. (“storage condition H4”), or room conditions and evaluating a measurable property. Changes in the measurable property over time are an indication of lack of stability with respect to that measurable property and the particular conditions studied. In some embodiments, the measurable property may change by less than about 50%, less than about 25%, less than about 20%, less than about 15%, less than about 10%, less than about 5%, less than about 1%, less than about 0.1%, or less than about 0.01% over a period of time of at least about 30 minutes, at least about 1 hours, at least about 6 hours, at least about 12 hours, at least about 24 hours, at least about 7 days, at least about two weeks, at least about one month, at least about three months, at least about six months, or at least about one year. Examples of measurable properties include, but are not limited to, whiteness, brightness, yellowness, clarity, opacity, gloss, size, smoothness, friability, transmission haze, adhesion, hardness, and moisture barrier.

### 3. Methods of Making Edible Coating Compositions

The methods of making the edible coating compositions may comprise mixing HPMC, a triester of citric acid, lecithin, and a C₁₂₋₂₄ fatty acid, in no particular order, to form a mixture, and optionally combining the mixture with water.

In general, the mixing and combining steps may be performed by any suitable method using any suitable apparatus known to one of skill in the art. Examples of suitable apparatuses for combining the ingredients of the compositions disclosed herein include, but are not limited to, plow mixers, low-shear overhead mixers in solution preparation, multi-action blades on overhead stirrers, and the like. Suitable mixers are available commercially from Littleford Day, Inc. (Florence, Ky.) and Lodige Mixers (Paderborn Germany), among others.

In one aspect, the method comprises: 1) weighing the desired amount of water into an appropriately sized container; 2) stirring the water with a high vortex; 3) mixing the dry ingredients and adding the dry ingredients into the vortex wall with rapid stirring; 4) stirring the resulting mixture at the rapid stirring rate for 5 minutes; and stirring the resulting mixture at a stirring rate that is 25% less than the rapid stirring rate for 25 minutes.

The compositions described herein can be manufactured using techniques and equipment that are known and commonly used in the art. Manufacturing steps such as order of component addition, mixing temperatures (heating and/or cooling), mixing time, mixing speed, etc. can be driven by either by formulation or equipment requirements, or both. A number of parameters can be modified during the manufacturing process without substantial effect on the efficacy of the resulting product. The manufacturing methods and processes can further include separate steps for validating the resulting composition (e.g., the total amounts, ratios, and even distribution of components in the composition, etc.).

The edible coating is easily prepared, which increases efficiency of coating, thus providing a more cost-effective coating. In some embodiments, the edible coating may be ready to use in less than about 90 minutes, less than about 85 minutes, less than about 80 minutes, less than about 75 minutes, less than about 70 minutes, less than about 65 minutes, less than about 60 minutes, less than about 55 minutes, less than about 50 minutes, less than about 45 minutes, less than about 40 minutes, less than about 35 minutes, or less than about 30 minutes.

### 4. Methods of Using Edible Coating Compositions

In one aspect, the edible coating composition may be applied directly or indirectly to an edible substrate to produce a coated edible substrate. The edible substrate may or may not be pre-coated with other coatings. In other words, in some embodiments no pre-coat or subcoat is needed—again, the edible coating composition may be applied directly to the edible substrate, food, pharmaceutical, or nutraceutical. In some embodiments, the edible substrate may be a nutraceutical or pharmaceutical dosage unit, such as a capsule, tablet or softgel. Other suitable edible substrates would be known to one of ordinary skill in the art.

The edible coating compositions and edible coatings may be used in food, pharmaceutical or nutraceutical applications intended for use in mammals, including, without limitation, rodents, canines, felines, non-human primates, ungulates, and humans. The edible coating compositions and edible coatings may be used to coat pharmaceutical or non-pharmaceutical dosage units.

The disclosed edible coating compositions may, at a suitable concentration which is system dependent, be applied (e.g., sprayed) to form an edible coating on an edible substrate. In certain embodiments, the edible coating compositions may be applied using commercially-available equipment. Equipment known in the industry for standard tablet coating are Thomas Engineering, Inc., Vector Corporation Hi-Coater®, O'Hara Technologies, and Coating Systems International TechniCota tablet coating systems.

In some embodiments, the methods may include applying an edible coating composition to an edible substrate to a weight gain of at least about 1%, at least about 2%, at least about 3%, at least about 4%, at least about 5%, at least about 6%, at least about 7%, at least about 8%, at least about 9%, or at least about 10%. In some embodiments, the methods may include applying an edible coating composition to a substrate to a weight gain of at most about 10%, at most about 9%, at most about 8%, at most about 7%, at most about 6%, at most about 5%, at most about 4%, at most about 3%, or at most about 2%. For example, the methods may include applying an edible coating composition to a substrate to a weight gain ranging from about 2% to about 6%.

In some embodiments, the film coating suspension may be applied to a substrate by loading substrate into a vented coating pan, such as, for example, a 15-inch, 24-inch, 48-inch, or 60-inch side-vented coating pan.

The inlet temperature may be at least about 45° C., at least about 50° C., at least about 55° C., at least about 58° C., at least about 60° C., at least about 65° C., and at least about 70° C. The inlet temperature may be less than about 80° C., less than about 75° C., less than about 70° C., less than about 65° C., and less than about 60° C. This includes, for example, about 50° C. to about 80° C., about 50° C. to about 65° C., or about 55° C. to about 75° C. The outlet temperature may be at least about 35° C., at least about 40° C., at least about 44° C. and at least about 45° C. The outlet temperature may be less than about 65° C., less than about 60° C., less than about 50° C., and less than about 46° C. This includes, for example, about 44° C. to about 65° C., about 44° C. to about 46° C., or about 45° C. to about 60° C.

The atomizing air pressure may be at least about 20 psi, at least about 21 psi, at least about 25 psi, at least about 30 psi, at least about 35 psi, at least about 40 psi, at least about 45 psi, at least about 50 psi, and at least about 55 psi. The atomizing air pressure may be less than about 75 psi, less than about 70 psi, less than about 65 psi, less than about 60 psi, less than about 55 psi, less than about 50 psi, less than about 40 psi, and less than about 30 psi. This includes, for example, about 20 to about 75 psi, about 23 to about 25 psi, about 25 to about 30 psi, about 35 to about 55 psi, or about 50 to about 70 psi.

In some embodiments, the spray rate may be at least about 10 g/min, at least about 15 g/min, at least about 20 g/min, at least about 25 g/min, at least about 30 g/min, at least about 35 g/min, at least about 40 g/min, at least about 50 g/min, at least about 75 g/min, at least about 100 g/min, at least about 125 g/min, at least about 150 g/min, at least about 175 g/min, at least about 200 g/min, at least about 225 g/min, at least about 250 g/min, at least about 275 g/min, at least about 300 g/min, at least about 325 g/min, at least about 350 g/min, at least about 375 g/min, at least about 400 g/min, at least about 425 g/min, at least about 450 g/min, at least about 475 g/min, at least about 500 g/min, at least about 525 g/min, at least about 550 g/min, or at least about 575 g/min. In some embodiments, the spray rate may be at most about 600 g/min, at most about 575 g/min, at most about 550 g/min, at most about 525 g/min, at most about 500 g/min, at most about 475 g/min, at most about 450 g/min, at most about 425 g/min, at most about 400 g/min, at most about 375 g/min, at most about 350 g/min, at most about 325 g/min, at most about 300 g/min, at most about 275 g/min, at most about 250 g/min, at most about 225 g/min, at most about 200 g/min, at most about 175 g/min, at most about 150 g/min, at most about 125 g/min, at most about 100 g/min, at most about 95 g/min, at most about 90 g/min, at most about 85 g/min, at most about 80 g/min, at most about 75 g/min, at most about 70 g/min, at most about 65 g/min, at most about 60 g/min, at most about 55 g/min, at most about 50 g/min, at most about 45 g/min, at most about 40 g/min, at most about 35 g/min, at most about 30 g/min, at most about 25 g/min, at most about 20 g/min, at most about 19 g/min, at most about 18 g/min, at most about 17 g/min, at most about 16 g/min, at most about 15 g/min, at most about 14 g/min, at most about 13 g/min, at most about 12 g/min, at most about 11 g/min, or at most about 10 g/min. This includes, for example, about 10 g/min to about 20 g/min, about 10 g/min to about 12 g/min, about 12 g/min to about 15 g/min, about 15 g/min to about 20 g/min, about 35 g/min to about 75 g/min, about 35 g/min to about 45 g/min, about 45 g/min to about 55 g/min, about 55 g/min to about 75 g/min, about 175 g/min to about 350 g/min, about 175 g/min to about 275 g/min, about 275 g/min to about 325 g/min, about 325 g/min to about 350 g/min, about 275 g/min to about 600 g/min, about 275 g/min to about 450 g/min, about 450 g/min to about 550 g/min, or about 550 g/min to about 600 g/min.

A higher spray rate may require the performance of a curing step after spray application. Without wishing to be limited by any particular theory, it is believed that a higher spray rate results in more water within the resulting coating. Therefore, without curing, the excess water within a coating produced by a higher spray rate may penetrate the core. In some embodiments, the methods may include curing the edible coating for at least about 1 minute, at least about 2 minutes, at least about 3 minutes, at least about 4 minutes, at least about 5 minutes, at least about 6 minutes, at least about 7 minutes, at least about 8 minutes, at least about 9 minutes, or at least about 10 minutes. In some embodiments, the methods may include curing the edible coating for at most about 30 minutes, at most about 25 minutes, at most about 20 minutes, at most about 15 minutes, at most about 14 minutes, at most about 13 minutes, at most about 12 minutes, at most about 11 minutes, at most about 10 minutes, at most about 9 minutes, at most about 8 minutes, at most about 7 minutes, at most about 6 minutes, at most about 5 minutes, at most about 4 minutes, at most about 3 minutes, or at most about 2 minutes. This includes embodiments where the methods include curing the edible coating for times ranging from about 1 minute to about 30 minutes, such as times ranging from about 2 minutes to about 20 minutes, or times ranging from about 5 minutes to about 15 minutes.

The coating time may be at least about 5 minutes, at least about 30 minutes, at least about 1 hour, at least about 2 hours, or at least about 3 hours. The coating time may be at most about 6 hours, at most about 3 hours, at most about 2 hours, at most about 1 hour, or at most about 30 minutes. The coating time may range from about 5 minutes to about 6 hours, such as from about 10 minutes to about 3 hours.

The film coating composition can be allowed to dry, forming a film coating on the substrate. Exemplary coating parameters are provided in Table 1.

TABLE 1

| Pan Size | 15 inch | 24 inch | 48 inch | 60 inch |
|---|---|---|---|---|
| Air volume (cfm) | 225-250 | 250-320 | 1800-2400 | 3800-4200 |
| Inlet temp. (° C.) | 50-65 | 50-65 | 55-75 | 55-80 |
| Outlet temp. (° C.) | 44-46 | 44-46 | 45-60 | 46-65 |
| Curing temp. (° C.)* | 42-44 | 42-44 | 42-50 | 42-50 |
| Pre-warm temp. (° C.) | 38-40 | 38-40 | 40-45 | 40-45 |
| Spray rate (g/min) | 10-20 | 35-75 | 175-350 | 275-600 |
| | 10-12** | 35-45** | 175-275** | 275-450** |
| | 12-15*** | 45-55*** | 275-325*** | 450-550*** |
| | 15-20**** | 55-75**** | 325-350**** | 550-600**** |
| Atomizing air pressure (psi) | 23-25 | 25-30 | 35-55 | 38-55 |
| Pattern air pressure (psi) | 20 | 20 | 35-50 | 35-50 |
| No. of guns | 1 | 1 | 3-4 | 4-6 |
| Pan speed (rpm) | 10-15 | 10-15 | 4-10 | 4-8 |
| Pan charge weight (kg) | 1-3 | 12-14 | 120-200 | 250-350 |

*Curing temperature if curing necessary or desired.
**Range of spray rate where curing is likely unnecessary to achieve moisture barrier properties.
***Range of spray rate where curing may be necessary to achieve moisture barrier properties.
****Range of spray rate where curing is likely necessary to achieve moisture barrier properties.

In some embodiments, a coating pan may be charged with a substrate such as, for example, capsules, tablets, and/or softgels. In some embodiments, the substrate may be warmed to about 33° C. to about 50° C. Further processing parameters may be as set forth herein.

### EXAMPLES

The following examples are presented to illustrate the present invention and to assist one of ordinary skill in making and using the same. The examples are not intended in any way to otherwise limit the scope of the invention.

Materials.

As used herein, the following terms have the corresponding meanings. “HPMC 15 SE” denotes Spectracel™ 15-SE (available commercially from Sensient Technologies, Inc., St. Louis, Mo.). “TEC” denotes triethyl citrate (available commercially from Penta International Corp., Livingston, N.J.). “Talc” denotes Talc Imperial 1889L (available commercially from Imerys Talc America, San Jose, Calif.). “Sodium bicarbonate” denotes sodium bicarbonate USP (available commercially from Church & Dwight, Green River, Wyo.). Sunflower lecithin (available commercially from Cargill, Inc., Minneapolis, Minn.), stearic acid (available commercially from Brenntag Mid-South, Henderson, Ky.), magnesium stearate (available commercially from Mallinckrodt Pharmaceuticals, Ireland), TiO₂ (available commercially from Huntsman Pigments, The Woodlands, Tex.), soya lecithin (available commercially from Cargill, Inc., Minneapolis, Minn.) and polysorbate 80 (available commercially from BASF Corp., Florham Park, N.J.) are denoted by their standard names. “PVA” denotes polyvinyl alcohol (available commercially from Shin-Etsu JVP, Japan VAM & POVAL CO., Ltd., Japan). “MCT” denotes Neobee® M-5 medium chain triglycerides (available commercially from Stepan Co., Northfield, Ill.) sodium citrate dihydrate, citric acid and Glycerol mono, di, tri (Imwitor 742—Cremer OLEO GmbH & Co.).

Unless otherwise indicated, example compositions were prepared by mixing the ingredients in a Littleford Plow Mixer equipped with a low-shear overhead mixer (available from Littleford Day, Inc., Florence, Ky.) according to the methods described herein.

### Example 1

| | % Film |
|---|---|
| | Coating |
| Material | Composition |
| HPMC 15 SE | 64.00 |
| TEC | 1.80 |
| Sunflower Lecithin | 4.20 |
| Stearic Acid | 30.00 |
| Total | 100 |

### Example 2

| | % Film |
|---|---|
| | Coating |
| Material | Composition |
| HPMC 15 SE | 55.00 |
| TEC | 5.00 |
| Sunflower Lecithin | 2.00 |
| Stearic Acid | 38.00 |
| Total | 100 |

### Example 3

| | % Film |
|---|---|
| | Coating |
| Material | Composition |
| HPMC 15 SE | 61.20 |
| TEC | 2.80 |
| Sunflower Lecithin | 5.40 |
| Stearic Acid | 30.60 |
| Total | 100 |

### Example 4

| | % Film |
|---|---|
| | Coating |
| Material | Composition |
| HPMC 15 SE | 70.00 |
| TEC | 1.00 |
| Sunflower Lecithin | 2.00 |
| Stearic Acid | 27.00 |
| Total | 100 |

### Example 5

| | % Film |
|---|---|
| | Coating |
| Material | Composition |
| HPMC 15 SE | 70.00 |
| TEC | 5.00 |
| Sunflower Lecithin | 10.00 |
| Stearic Acid | 15.00 |
| Total | 100 |

### Example 6

| | % Film |
|---|---|
| | Coating |
| Material | Composition |
| HPMC 15 SE | 70.00 |
| TEC | 1.00 |
| Sunflower Lecithin | 10.00 |
| Stearic Acid | 19.00 |
| Total | 100 |

### Example 7

| | % Film |
|---|---|
| | Coating |
| Material | Composition |
| HPMC 15 SE | 70.00 |
| TEC | 5.00 |
| Sunflower Lecithin | 2.00 |
| Stearic Acid | 23.00 |
| Total | 100 |

### Example 8

| | % Film |
|---|---|
| | Coating |
| Material | Composition |
| HPMC 15 SE | 55.00 |
| TEC | 5.00 |
| Sunflower Lecithin | 10.00 |
| Stearic Acid | 30.00 |
| Total | 100 |

### Example 9

| | % Film |
|---|---|
| | Coating |
| Material | Composition |
| HPMC 15 SE | 55.00 |
| TEC | 1.00 |
| Sunflower Lecithin | 10.00 |
| Stearic Acid | 34.00 |
| Total | 100 |

### Example 10

| | % Film |
|---|---|
| | Coating |
| Material | Composition |
| HPMC 15 SE | 55.00 |
| TEC | 3.00 |
| Sunflower Lecithin | 2.00 |
| Stearic Acid | 40.00 |
| Total | 100 |

### Example 11

| | % Film |
|---|---|
| | Coating |
| Material | Composition |
| HPMC 15 SE | 55.00 |
| TEC | 1.00 |
| Sunflower Lecithin | 4.00 |
| Stearic Acid | 40.00 |
| Total | 100 |

### Example 12

| | % Film |
|---|---|
| | Coating |
| Material | Composition |
| HPMC 15 SE | 57.00 |
| TEC | 1.00 |
| Sunflower Lecithin | 2.00 |
| Stearic Acid | 40.00 |
| Total | 100 |

### Example 13

| | % Film |
|---|---|
| | Coating |
| Material | Composition |
| HPMC 15 SE | 70.00 |
| TEC | 1.80 |
| Sunflower Lecithin | 4.20 |
| Stearic Acid | 24.00 |
| Total | 100 |

### Example 14

| | % Film |
|---|---|
| | Coating |
| Material | Composition |
| HPMC 15 SE | 50.880 |
| TEC | 1.431 |
| Sunflower Lecithin | 3.339 |
| Stearic Acid | 23.850 |
| TiO₂ | 20.000 |
| Sodium Bicarbonate | 0.500 |
| Total | 100 |

### Example 15

| | % Film |
|---|---|
| | Coating |
| Material | Composition |
| HPMC 15 SE | 52.670 |
| Stearic Acid | 24.000 |
| TiO₂ | 18.000 |
| Citric Acid Anhydrous | 0.500 |
| Sodium Citrate Dihydrate | 0.800 |
| Glycerides, Mono, Di and Tri | 1.200 |
| Sunflower Lecithin | 2.380 |
| TEC | 0.450 |
| Total | 100 |

### Example 16

| | % Film |
|---|---|
| | Coating |
| Material | Composition |
| HPMC 15 SE | 64.00 |
| Stearic Acid | 30.00 |
| Sunflower Lecithin | 4.20 |
| TEC | 1.80 |
| Total | 100 |

### Comparative Example 1

| | % Film |
|---|---|
| | Coating |
| Material | Composition |
| PVA | 31.64 |
| Talc | 34.09 |
| TiO₂ | 32.00 |
| Xanthan Gum | 0.45 |
| Soya Lecithin | 0.46 |
| Total | 100 |

### Comparative Example 2

| | % Film |
|---|---|
| | Coating |
| Material | Composition |
| PVA | 40.00 |
| Magnesium Stearate | 15.00 |
| Stearic Acid | 23.70 |
| TiO₂ | 15.00 |
| Xanthan Gum | 0.30 |
| Polysorbate 80 | 1.00 |
| MCT | 5.00 |
| Total | 100 |

### Comparative Example 3

| | % Film |
|---|---|
| | Coating |
| Material | Composition |
| HPMC 15 SE | 55.00 |
| TEC | 7.00 |
| Sunflower Lecithin | 0.00 |
| Stearic Acid | 38.00 |
| Total | 100 |

### Comparative Example 4

| | % Film |
|---|---|
| | Coating |
| Material | Composition |
| HPMC 15 SE | 55.00 |
| TEC | 0.00 |
| Sunflower Lecithin | 7.00 |
| Stearic Acid | 38.00 |
| Total | 100 |

### Example 17

### Moisture Uptake

Placebos consisting of arginine cores were coated to a 3% and 5% weight gain with Example 1 and Comparative Examples 1 and 2. The coated placebos were weighed and measured for width after coating.

The coated placebos were then exposed to storage condition H2 or storage condition H4. Weight and width of the coated placebos were measured after exposure to the conditions for 8 hours, 16 hours, 24 hours and 32 hours. The placebos coated to a 3% weight gain with Example 1 were exposed to storage conditions H2 and H4 for 3 months after which weight and width were measured. The weight measurements (values in g) are shown in Table 2 and the width measurements (values in inches) are shown in Table 3.

TABLE 2

| Sample | | 0 hrs. | 8 hrs. | 16 hrs. | 24 hrs. | 32 hrs. | 3 mos. |
|---|---|---|---|---|---|---|---|
| Ex. 1 - | H2 | 1.2688 | 1.2903 | 1.3044 | 1.3246 | 1.3376 | 1.4065 |
| 3% | H4 | 1.2536 | 1.3509 | 1.322 | 1.435 | 1.4358 | 1.467 |
| Ex. 1 - | H2 | 1.3036 | 1.3152 | 1.4172 | 1.3319 | 1.3375 | n/a |
| 5% | H4 | 1.306 | 1.3626 | 1.409 | 1.46 | 1.4698 | n/a |
| C. Ex. | H2 | 1.2772 | 1.2086 | 1.2815 | 1.2839 | 1.2842 | n/a |
| 1 - 3% | H4 | 1.2885 | 1.4265 | Broke open after 8 hours | | | |
| C. Ex. | H2 | 1.3021 | 1.3044 | 1.3048 | 1.3068 | 1.3068 | n/a |
| 1 - 5% | H4 | 1.3081 | 1.3901 | Broke open after 8 hours | | | |
| C. Ex. | H2 | 1.283 | 1.2852 | 1.2854 | 1.2878 | 1.2883 | n/a |
| 2 - 3% | H4 | 1.285 | 1.341 | 1.3076 | 1.4513 | 1.4523 | n/a |
| C. Ex. | H2 | 1.3067 | 1.3078 | 1.4146 | 1.3092 | 1.309 | n/a |
| 2 - 5% | H4 | 1.3316 | 1.3601 | 1.3918 | 1.4349 | 1.4673 | n/a |

TABLE 3

| Sample | | 0 hrs. | 8 hrs. | 16 hrs. | 24 hrs. | 32 hrs. | 3 mos. |
|---|---|---|---|---|---|---|---|
| Ex. 1 - | H2 | 0.323 | 0.3271 | 0.3306 | 0.335 | 0.3371 | 0.466 |
| 3% | H4 | 0.323 | 0.3375 | 0.3321 | 0.3575 | 0.3575 | 0.463 |
| Ex. 1 - | H2 | 0.3292 | 0.32 | 0.3578 | 0.3326 | 0.334 | n/a |
| 5% | H4 | 0.3305 | 0.3385 | 0.3465 | 0.3565 | 0.3575 | n/a |
| C. Ex. | H2 | 0.3235 | 0.324 | 0.324 | 0.3248 | 0.3248 | n/a |
| 1 - 3% | H4 | 0.3308 | 0.489 | Broke open after 8 hours | | | |
| C. Ex. | H2 | 0.326 | 0.326 | 0.3271 | 0.3271 | 0.3271 | n/a |
| 1 - 5% | H4 | 0.327 | 0.336 | Broke open after 8 hours | | | |
| C. Ex. | H2 | 0.328 | 0.328 | 0.328 | 0.328 | 0.328 | n/a |
| 2 - 3% | H4 | 0.3281 | 0.337 | 0.3318 | 0.3665 | 0.3665 | n/a |
| C. Ex. | H2 | 0.3317 | 0.3325 | 0.3551 | 0.3328 | 0.333 | n/a |
| 2 - 5% | H4 | 0.3395 | 0.3415 | 0.349 | 0.357 | 0.365 | n/a |

### Example 18

### Moisture Uptake

Placebos coated with Example 1 to a 3% and 5% weight gain were exposed to storage condition H4 for 30 minutes in a VTI-SA Sorption Analyzer (available commercially from TA Instruments, New Castle, Del.). The placebos coated with Example 1 to a 3% weight gain showed a moisture uptake of ˜0.3% and the placebos coated with Example 1 to a 5% weight gain showed a moisture uptake of ˜0.4%.

### Example 19

### Moisture Uptake

The Examples of the disclosure were compared to commercially available coatings, (e.g., BASF Kollicoat Protect; Aqualon Aquarius MG; and Colorcon Opadry AMB, all at 4% wt. gain of coating), in their ability to withstand moisture uptake. Commercially available samples were exposed to 25° C., 75% Room Humidity for 7 days, wherein all commercial samples demonstrated at least swelling of the tablet, with BASF exhibiting softening and Aqualon exhibiting bursting of tablet. In contrast, Example 19 demonstrates negligible swelling or adverse effects at 40° C., 75% Room Humidity for 60 days, for both a 2% and 3% wt. gain of coating. These results demonstrate the superior moisture stability of the Example, even when exposed to more rigorous conditions.

### Example 20

### Slip

Placebos were coated to a 3% weight gain with a traditional HPMC coating (76% Spectracel 6 SE, available commercially from Sensient Technologies, Inc., St. Louis, Mo., 4% deionized water, and 20% USP-grade glycerine) or to a 3% or 5% weight gain with the composition of Example 1. The slip was measured by utilizing a TA.XT.Plus Texture Analyzer, as described above, and the average of measured slip values is reported below in Table 4. However, samples were tested as low as 22 g of friction force comparatively.

TABLE 4

| Sample | Avg. Slip (in g) |
|---|---|
| 3% - Trad. HPMC Coating | 94 |
| 3% - Example 1 Coating | 31.9 |
| 5% - Example 1 Coating | 27.1 |

### Example 21

### Friability

Placebos were coating to 3% and 5% weight gain with Example 1. Friability was testing using Dr. Schleuniger Pharmatron method FTV-2, as described above. The placebos coated to a 3% and 5% weight gain had a measured friability of 0.00%.

### Example 22

### Maintaining Hardness

Placebos with an average hardness of 46.5 kp were coated to a 3% weight gain with Example 1. The resulting coated tablets had an average hardness of 46.5 kp.

### Example 23

### Enhancing Hardness

Arginine placebos with an average hardness of 22.4 kp were coated to a 3% weight gain with Example 1. The resulting coated tablets had an average hardness of 45.0 kp.

### Example 24

### Disintegration

Immediate release placebos were coated with Example 1 at 2%, 3%, 4% and 5% weight gain. The coated placebos were exposed to 37° C. deionized water in a Dr. Schleuniger Pharmatron DT2-IS. The placebos coated with Example 1 at 2% weight gain dissolved in 2 minutes 15 seconds, the placebos coated with Example 1 at 3% weight gain dissolved in 2 minutes, 25 seconds, the placebos coated with Example 1 at 4% weight gain dissolved in 3 minutes, 55 seconds, and the placebos coated with Example 1 at 5% weight gain dissolved in 5 minutes, 5 seconds.

### Example 25

### Powder Rheology

The compositions of the disclosure may be in powder form and have superior rheology properties. The Basic Flowability Energy, BFE, Stability Index SI, Specific Energy, SE, and flow rate index, FRI, are energy measurements required to establish a particular flow pattern in a conditioned, precise volume of powder. The flow properties are influenced by particle size, shape, texture, stiffness, cohesivity, density, electrostatic charge, moisture content, elasticity, porosity, friability and surface additive. The shape of the curve may be horizontally even and then curve consistently upwards when the tip speed is reduced. The results show that the present compositions may have consistent powder flowability properties. This influences production, packaging and final use by a customer.

Spectrablend MBH WHITE Powder Rheology was taken on the FT4 Powder Rheometer—Freeman technology. Results are displayed in Table 5.

The Basic Flowability Energy, BFE, is the energy required to establish a particular flow pattern in a conditioned, precise volume of powder. The BFE interprets the flow properties influenced by particle size, shape, texture, stiffness, cohesivity, density, electrostatic charge, moisture content, elasticity, porosity, friability and surface additive. The shape of the curve may be horizontally even and then curve consistently upwards when the tip speed is reduced. The results show that the compositions have consistent flow ability properties.

The Stability Index, SI, is a measure of how stable the powder is, meaning that it determines if the powder will undergo segregation over time. Results for a stable SI value may be small changes (0.9<SI<1.1). The compositions may have values that fall within the expected SI range, indicating that compositions are robust and will not undergo substantial product segregation on packaging and storage. Robust material may have an SI value of ˜1. This value is also depicted by the straight line seen in the BFE testing.

The Specific Energy, SE, measurement is the flow performance of a powder in a low stress environment and is indicative of the low tip speed. This may have a consistent upward trending slope in the graph. The data values of an SE<5 may suggest that the powder has low cohesion properties. An SE value of 5<SE<10 suggests that the powder is moderately cohesive, and an SE value of >10 suggests that the powder has high cohesive properties. SBMBH White has moderate cohesive properties according to the data.

The flow rate index, FRI, provides the basic flow rate of the powder. FRI data of >3 are powders having cohesiveness. FRI data of 1.5<FRI<3.0 are powders with an average flow rate sensitivity and most powders fall in this range. FRI data of ˜1, are powder with large particles. FRI data of <1.0 may show pseudo plastic or Newtonian flow rate, which is usually seen for powders containing flow enhancers like talc. The SBMBH White FRI data shows that the flow ability is within the typical powder flow ability range.

Conditioned Bulk Density, CBD, is the bulk density of the powder.

TABLE 5

| Material | BFE, | | | SE, | CBD, |
|---|---|---|---|---|---|
| and Batch | mJ | SI | FRI | mJ/g | g/ml |
| SBMBH | 871.2198 | 1.047448 | 1.627075 | 6.817292 | 0.62875 |
| WHITE/ | | | | | |
| 92-122 | | | | | |
| RUN 1 | | | | | |
| SBMBH | 876.8405 | 0.983524 | 1.441885 | 6.348587 | 0.619625 |
| WHITE/ | | | | | |
| 92-125 | | | | | |
| RUN 2 | | | | | |
| SBMBH | 855.9967 | 0.905764 | 1.931297 | 7.045692 | 0.596 |
| WHITE/ | | | | | |
| 92-126 | | | | | |
| RUN 3 | | | | | |

Additionally, the particle size distribution of the powder exhibited a d(0.5) is 74.66 microns, d(0.9) is 201.60 microns.

What is claimed is:

1. An edible coating composition comprising:
hydroxypropyl methylcellulose;
a triester of citric acid;
lecithin; and
a C₁₂₋₂₄ fatty acid,
wherein the C₁₂₋₂₄ fatty acid is present in an amount of less than about 32.5% by weight of the non-water components of the composition, or
wherein the C₁₂₋₂₄ fatty acid is present in an amount of from about 32.5% to about 50% by weight of the non-water components of the composition and the triester of citric acid is present in an amount of greater than about 4% by weight of the non-water components of the composition.

2. An edible coating composition comprising:
about 50.0% to about 75.0% by weight hydroxypropyl methylcellulose;
about 1.0% to about 6.0% by weight triester of citric acid;
about 0.5% to about 10.0% by weight of lecithin; and
about 15.0% to about 45.0% by weight of a C₁₂₋₂₄ fatty acid,
wherein all percentages by weight are of non-water components of the composition, and
wherein applying the composition to an edible substrate to a weight gain of about 5% compared to the edible substrate produces an edible substrate coated with an edible coating, the edible coating having a moisture uptake of less than about 1% when exposed to 40° C. and 75% relative humidity for 30 minutes as measured by a VTI-SA Sorption Analyzer.

3. The composition of claim 1, wherein the hydroxypropyl methylcellulose is present in an amount of from about 40% to about 80% by weight of the non-water components of the composition.

4. The composition of any of claim 1, wherein the hydroxypropyl methylcellulose is present in an amount of from about 50% to about 75% by weight of the non-water components of the composition.

5. The composition of any of claims 1-4, wherein the hydroxypropyl methylcellulose is present in an amount of from about 55% to about 70% by weight of the non-water components of the composition.

6. The composition of any of claims 1-5, the hydroxypropyl methylcellulose having a viscosity in 2% aqueous solution at 20° C. of from about 12.0 to about 18.0 centipoise.

7. The composition of any of claim 1 or 3-6, wherein the triester of citric acid is present in an amount of from about 0.1% to 10.0% by weight of the non-water components of the composition.

8. The composition of any of claims 1-7, wherein the triester of citric acid is present in an amount of from about 1.0% to about 3.0% by weight of the non-water components of the composition.

9. The composition of any of claims 1-7, wherein the triester of citric acid is present in an amount of from about 4.0% to about 6.0% by weight of the non-water components of the composition.

10. The composition of any of claims 1-9, wherein the triester of citric acid comprises at least one of triethyl citrate, tripropyl citrate, tributyl citrate, acetyl triethyl citrate, acetyl tributyl citrate, and combinations thereof.

11. The composition of any of claims 1-10, wherein the triester of citric acid comprises triethyl citrate.

12. The composition of any of claim 1 or 3-11, wherein the lecithin is present in an amount of from about 0.1% to about 15% by weight of the non-water components of the composition.

13. The composition of any of claim 1 or 3-12, wherein the lecithin is present in an amount of from about 0.5% to about 10% by weight of the non-water components of the composition.

14. The composition of any of claims 1-13, wherein the lecithin is present in an amount of from about 1% to about 5% by weight of the non-water components of the composition.

15. The composition of any of claims 1-14, wherein the lecithin comprises at least one of sunflower lecithin, soy lecithin, egg lecithin, peanut lecithin, sesame lecithin, canola lecithin, and combinations thereof.

16. The composition of any of claims 1-15, wherein the lecithin comprises sunflower lecithin.

17. The composition of any of claim 1 or 3-16, wherein the C₁₂₋₂₄ fatty acid is present in an amount of from about 10% to about 50% by weight of the non-water components of the composition.

18. The composition of any of claims 1-17, wherein the C₁₂₋₂₄ fatty acid is present in an amount of from about 25% to about 40% by weight of the non-water components of the composition.

19. The composition of any of claims 1-18, wherein the C₁₂₋₂₄ fatty acid comprises at least one of stearic acid, oleic acid, palmitic acid, arachidic acid, myristic acid, and combinations thereof.

20. The composition of any of claims 1-19, wherein the C₁₂₋₂₄ fatty acid comprises stearic acid.

21. The composition of any of claims 1-20, wherein the ratio of hydroxypropyl methylcellulose to C₁₂₋₂₄ fatty acid is from about 1:1 to about 5:1.

22. The composition of any of claims 1-21, wherein the ratio of C₁₂₋₂₄ fatty acid to triester of citric acid is from about 5:1 to about 20:1.

23. The composition of any of claims 1-22, wherein applying the composition to an edible substrate produces a coated edible substrate, the edible substrate and the coated edible substrate each having a reflectance spectrum having a lightness, whiteness and yellowness, wherein the lightness, whiteness or yellowness varies by less than about 50% from edible substrate to coated edible substrate.

24. The composition of any of claims 1-23, wherein applying the composition to an edible substrate produces a coated edible substrate, the edible substrate and the coated edible substrate each having a reflectance spectrum having a peak wavelength, a peak width and an integrated area, wherein the peak wavelength, peak width or integrated area varies by less than about 50% from edible substrate to coated edible substrate.

25. The composition of any of claims 1-24, wherein applying the composition to an edible substrate produces a coated edible substrate, the edible substrate and the coated edible substrate each having a gloss, wherein the gloss of the coated edible substrate is from about 0% to about 500% greater than the gloss of the edible substrate.

26. The composition of any of claims 1-25, wherein applying the composition to an edible substrate produces an edible coating on the edible substrate, the edible coating having a transmission haze of less than about 50% as measured by ASTM D1003-95, “Standard Test Method for Haze and Luminous Transmittance of Transparent Plastics” or a reflection haze of less than about 50% as measured by ASTM E430-91, “Standard Test Methods for Measurement of Gloss of High-Gloss Surfaces by Goniophotometry.”

27. The composition of any of claims 1-26, wherein applying the composition to an edible substrate to a weight gain of about 5% produces a coated edible substrate having a moisture uptake over exposure to 40° C. and 75% relative humidity for 30 minutes of less than about 0.5% as measured by a VTI-SA Sorption Analyzer.

28. The composition of any of claim 27, wherein the composition is applied to the edible substrate to a weight gain of about 3%.

29. The composition of any of claims 1-28, wherein the triester of citric acid is triethyl citrate and the triethyl citrate is present in an amount of from about 1% to about 3% by weight of the non-water components of the composition, hydroxypropyl methylcellulose is present in an amount of from about 60% to about 70% by weight of the non-water components of the composition, lecithin is sunflower lecithin and the sunflower lecithin is present in an amount of from about 2% to about 6% by weight of the non-water components of the composition, and the C₁₂₋₂₄ fatty acid is stearic acid and the stearic acid is present in an amount of from about 25% to about 35% by weight of the non-water components of the composition.

30. A coated edible substrate comprising a food, pharmaceutical or nutraceutical having an edible substrate having the composition of any of claims 1-29 applied thereon.

31. A method of using the coating composition of any of claims 1-29, the method comprising applying the composition to an edible substrate to form an edible coating thereon.

32. An edible coating composition of any of claims 1-29 further comprising an opacifying agent.

33. The edible coating composition of any of claim 32, wherein the opacifying agent comprises titanium dioxide.

34. A method of making a coating composition, the method comprising:
combining hydroxypropyl methylcellulose, a triester of citric acid, lecithin, and a C₁₂₋₂₄ fatty acid to form a mixture; and
optionally combining the mixture with water.

35. A coated edible substrate comprising a food, pharmaceutical or nutraceutical having an edible coating applied thereto, the edible coating comprising:
hydroxypropyl methylcellulose;
a triester of citric acid;
lecithin; and
a C₁₂₋₂₄ fatty acid,
wherein the C₁₂₋₂₄ fatty acid is present in an amount of less than about 32.5% by weight of the coating, or
wherein the C₁₂₋₂₄ fatty acid is present in an amount of from about 32.5% to about 50% by weight of the coating and the triester of citric acid is present in an amount of greater than about 4% by weight of the coating.

36. A coated edible substrate comprising a food, pharmaceutical or nutraceutical having an edible coating applied thereto, the edible coating comprising:
about 50.0% to about 75.0% by weight hydroxypropyl methylcellulose;
about 1.0% to about 6.0% by weight triester of citric acid;
about 0.5% to about 10.0% by weight of lecithin; and
about 15.0% to about 45.0% by weight of a C₁₂₋₂₄ fatty acid,
wherein all percentages by weight are of the coating, and
wherein the edible coating is applied to a weight gain of about 3% compared to the food, pharmaceutical or nutraceutical, and the edible coating has a moisture uptake of less than about 0.5% when exposed to 40° C. and 75% relative humidity for 30 minutes as measured by a VTI-SA Sorption Analyzer.
